(19) United States
(12) Patent Application Publication
(10) Pub. No.: US 2025/0185906 A1
(43) Pub. Date: Jun. 12, 2025

(54) PERIMETER WITH ADJUSTABLE LENS

(71) Applicant: Haag-Streit AG, Köniz (CH)

(72) Inventors: Peter STALDER, Konolfingen (CH); Philipp GLOOR, Wünnewil (CH)

(73) Assignee: Haag-Streit AG, Köniz (CH)

(21) Appl. No.: 18/837,374

(22) PCT Filed: Mar. 29, 2022
(86) PCT No.: PCT/EP2022/058230

§ 371 Date: Aug. 9, 2024

(51) Int. Cl. A61B 3/024; G02B 26/08; G02F 1/29

(52) U.S. Cl. CPC A61B 3/024; G02B 26/0816; G02F 1/294

(57) ABSTRACT

The perimeter includes a screen, a light source assembly, projection optics, and an adjustable beam deflector. The projection optics includes an adjustable lens having a focal length that can be changed by the control unit of the device. The projection optics further includes a projection lens element. The focal length of the adjustable lens is being changed as a function of the location of the stimulus light spot, thereby compensating for the location-dependent length of the optical path from the projection optics to the screen. The distance between the projection lens element and the adjustable lens is equal to the focal length of the projection lens element, which makes the spot size independent on the location.

### TECHNICAL FIELD

The present invention relates to a perimeter as well as to a method for operating the same.

In this context, a perimeter is a used to examine a patient's subjective visual field.

### BACKGROUND ART

A perimeter, as e.g. described in U.S. Pat. No. 4,561,738 or 7,475,987, comprises a light source, an adjustable deflector, as well as a screen. The adjustable deflector is used to generate a stimulus light spot at a desired location on the screen in order to assess the sensitivity of a patient's field of view for a particular spatial direction.

Typically, perimetry devices of this type face the challenge that the optical path length for the light depends on the coordinates where the stimulus light spot is to appear on the screen.

For example, for a spherical screen as it is e.g. shown in U.S. Pat. No. 4,561,738, the patient's eye is typically located at the spherical center of the screen, and the deflector, such as a set of movable mirrors, is therefore located off-axis. Hence, the length of the path the light has to travel from the light source via the deflector to the screen varies with the selected screen location.

On the other hand, the projected light spot should have substantially the same brightness and size for all screen locations. Hence, compensation optics are required. For example, EP1380251A2 describes using a path length correction implemented with displaceable mirrors. This is mechanically complex, though, and actuating the mirrors leads to a noise that can alert the patient of a new stimulus, which in turn can affect the accuracy of the examination.

### DISCLOSURE OF THE INVENTION

Hence, the present invention aims to solve the problem of providing a perimeter and a method of this type where the size of the stimulus light spot can be corrected in a simple matter.

This problem is solved by the perimeter and method of the independent claims.

Accordingly, the perimeter comprises at least the following elements:
A screen: The screen is used to receive the stimulus light spot for the patient.
A light source assembly: The light source assembly is adapted to generate visible light to be used for the stimulus.
Projection optics: The projection optics processes the light from the light source and is adapted to project the stimulus light spot onto the screen.
An adjustable deflector: The adjustable deflector is adapted to deflect the light in order to generate the stimulus light spot at a plurality of selectable coordinates on the screen.

According to the invention, the projection optics comprises an adjustable lens with adjustable focal length. This lens can be used to correct the focus of the stimulus light spot quickly and easily, and it may be controlled as a function of the path length of the light from the light source to the screen.

Advantageously, the projection optics comprises a projection lens element arranged coaxially with and at a distance from the adjustable lens. This lens element can support the imaging operation of the adjustable lens. It advantageously has a fixed focal length.

In particular, the projection lens element may be a positive lens. This may e.g. allow operating the adjustable lens at a large (positive or negative) focal length.

In that case, the focal length of the projection lens element is advantageously equal, within ±20%, to the axial distance between the adjustable lens and the projection lens element. As shown below, this ensures that the size of the stimulus light spot on the screen is substantially independent of the focal length used for the adjustable lens.

In another advantageous embodiment, the aperture of the combination of the projection optics and the deflector is chosen such that any light beam from the light source traversing the adjustable lens arrives at the screen. This ensures that the total amount of light in the stimulus does not change when varying the focal length of the adjustable lens.

The light source assembly typically forms a field stop that defines an illuminated area projected by the projection optics onto the screen. The field stop is advantageously illuminated homogeneously (e.g. with a homogeneity better than 25%). Hence, the diameter of the field stop, together with the magnification of the projection optics, defines the diameter of the stimulus light spot.

The field stop may e.g. be formed by a diaphragm in a Kohler illumination, or it may e.g. by the output aperture of a multi-mode light guide that homogenizes the light from the light source.

Advantageously, the diameter of the field stop can be varied, e.g. by using an adjustable diaphragm for defining the field stop.

The perimeter may further comprise a control unit connected to the adjustable lens and the adjustable deflector for controlling them.

This control unit is advantageously adapted to carry out at least the following steps:
Positioning the stimulus light spot on the screen at a plurality of locations by means of adjusting the adjustable deflector. As explained, the optical path length from the light source assembly to the location of the stimulus light spot varies between at least some of said locations.
Adjusting the focal length of the adjustable lens as a function of the path length.

This scheme provides an automatic compensation of the varying path length.

Advantageously, the control unit adjusts the focal length of the adjustable lens such that, for all said locations, the projection optics projects the field stop onto the screen. In other words, the field stop and the screen are kept in conjugate planes of the projection optics for the presently selected location.

In this case, the projection optics is advantageously designed to have the same magnification for all said locations, within an accuracy of 20%.

The adjustable deflector may comprise a mirror, which is advantageously located radially offset from the center axis of the screen in order to provide a large field of view to the patient.

In a simple design, the adjustable deflector comprises a mirror that can be titled around a first axis and a second axis, with the two axes being transversal to each other. This allows using a single mirror for two-dimensionally adjusting the location of the stimulus light spot on the screen.

Advantageously, for a computationally simple control, the first and second axes are perpendicular to each other, and/or they intersect.

In a simple design, the adjustable deflector comprises:
A first rotatory actuator, such as a stepper motor, with a first shaft rotatable around first axis.
A second rotatory actuator, such as a stepper motor, with a second shaft rotatable around the second axis.
A first coupler that couples the mirror and the first shaft. It rotationally locks the mirror to the first shaft for rotations about the first axis but allows the mirror to tilt about a third axis transversal to the first axis. In other words, when the first shaft rotates, the mirror rotates with it, but it is allowed to tilt around the third axis.
A second coupler that couples the mirror and the second shaft. It rotationally locks the mirror to the second shaft for rotations about the second axis but allows the mirror to tilt about a fourth axis transversal to the second axis. In other words, when the second shaft rotates, the mirror rotates with it, but it is allowed to tilt around the fourth axis.

In particular, the third axis rotates with the first shaft around the first axis and the fourth axis rotates with the second shaft around the second axis.

In particular, the third axis is perpendicular to the first axis and the fourth axis is perpendicular to the second axis.

The screen of a perimeter is typically rotationally symmetric to a screen axis. In order provide a wide field of view for the patient, the first and second rotatory actuator are advantageously arranged radially outside (in respect to the screen axis) from the mirror, i.e. the mirror is closer to the screen axis than the first and second rotatory actuators.

The invention also relates to a method for operating such a perimeter. This method comprises the step of positioning a stimulus light spot on the screen at a plurality of locations by means of adjusting the adjustable deflector.

Advantageously, and as mentioned, the optical path length from the light source assembly to the location varies between at least some of said locations. In this case, the method advantageously comprises the step of adjusting the focal length of the adjustable lens as a function of the path length.

In particular, for the reasons mentioned above, the method may comprise the step of adjusting the focal length of the adjustable lens such that, for all said locations, the projection optics projects a field stop of the light source assembly onto the screen.

### BRIEF DESCRIPTION OF THE DRAWINGS

The invention will be better understood and objects other than those set forth above will become apparent when consideration is given to the following detailed description thereof. Such description makes reference to the annexed drawings, wherein:

FIG. 1 shows a schematic view of an embodiment of a perimeter and a patient using it,

FIG. 2 shows a sectional view of the perimeter along a vertical plane through the central axis of the screen,

FIG. 3 shows the optical components generating the stimulus light spot on the screen,

FIG. 4 shows an embodiment of the light deflector,

FIG. 5 shows an embodiment of the light source assembly, the adjustable lens, and the projection lens element, and

FIG. 6 illustrates the distances between the field stop, the adjustable lens, the projection lens element, and the screen along the optical axis of the system.

### MODES FOR CARRYING OUT THE INVENTION

### Definitions

A “lens” as defined herein is a simple lens or a compound lens.

A “compound lens” comprises several simple lenses arranged coaxially, with the distance between the lenses being much smaller than the focal length of the lenses. Advantageously, the simple lenses of a compound lens are arranged axially adjacent to each other, i.e. they may touch. As known to the skilled person, the simple lenses of a compound lens may e.g. have different dispersion properties for generating an achromatic overall lens effect.

A “lens element” may be a lens and/or a curved mirror with transfer properties that can be approximated by means of a ray transfer matrix of the type

$$[ \begin{matrix}1 & 0 \\ - \frac{1}{f} & 1\end{matrix} ]$$

with f being the focal length of the lens element.

A “controllable liquid lens” is a lens the focal length of which depends on the curvature of the interface of at least one liquid. This curvature can be changed by means of an electrical field and/or by mechanical means. Advantageously, the curvature is changed by an electrical field applied to the liquid.

The term “axial” relates to the center line of the light beam from the light source assembly as it travels through the projection optics.

The term “axial distance” expresses the distance between two components along the axis of the light beam.

### Perimeter Overview

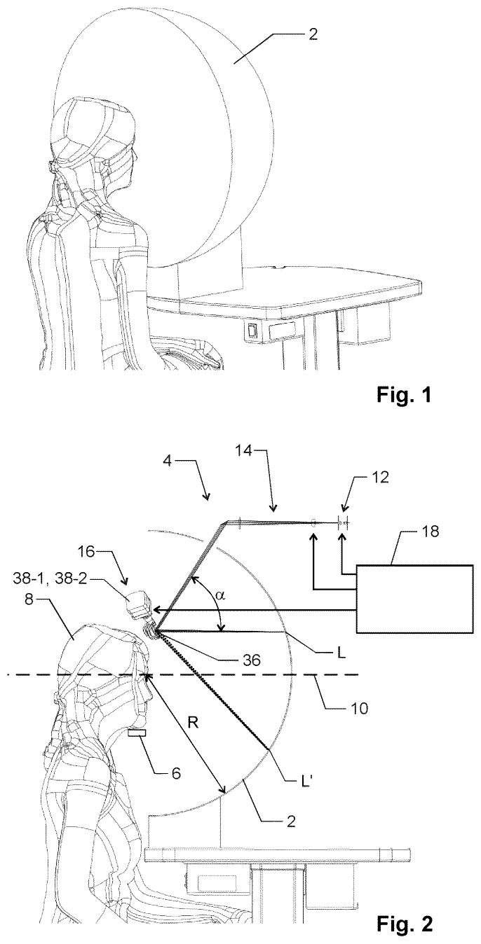

FIGS. 1 and 2 show a perimeter having a screen 2 as well as a projection system 4 for projecting a stimulus light spot to a location L, L′ on screen 2.

The perimeter further comprises a chin or headrest 6 for placing the head of a patient 8, and in particular the patient's eye, at a defined location in front of the screen.

As known to the skilled person, screen 2 of modern perimeters is typically curved having a central screen axis 10, and the patient's eye is advantageously placed close to or at an intersection with screen axis 10.

Advantageously, screen 2 is spherical, and screen axis 10 extends through the center point of the sphere defined by screen 2.

Projection system 4 comprises a light source assembly 12, projection optics 14, and an adjustable deflector 16. A control unit 18 controls light source assembly 12, projection optics 14, and beam deflector 16 to generate the stimulus light spot at the plurality of locations L, L′ on screen 2.

In order to perform a perimetry examination, control unit 18 sets beam deflector 16 to a desired location L and adjusts projection optics 14 accordingly, as described in more detail below. Then it operates light source assembly 12 to generate a stimulus light spot of defined brightness and size at that location L. The brightness of the stimulus is varied to determine the visual threshold. This operation is repeated for a plurality of locations.

### Projection System

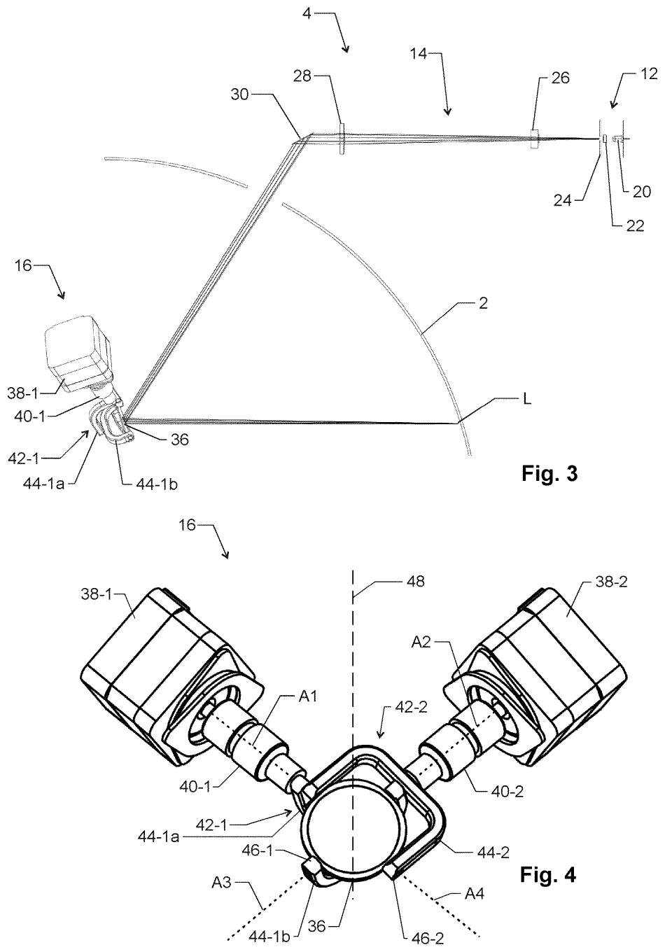

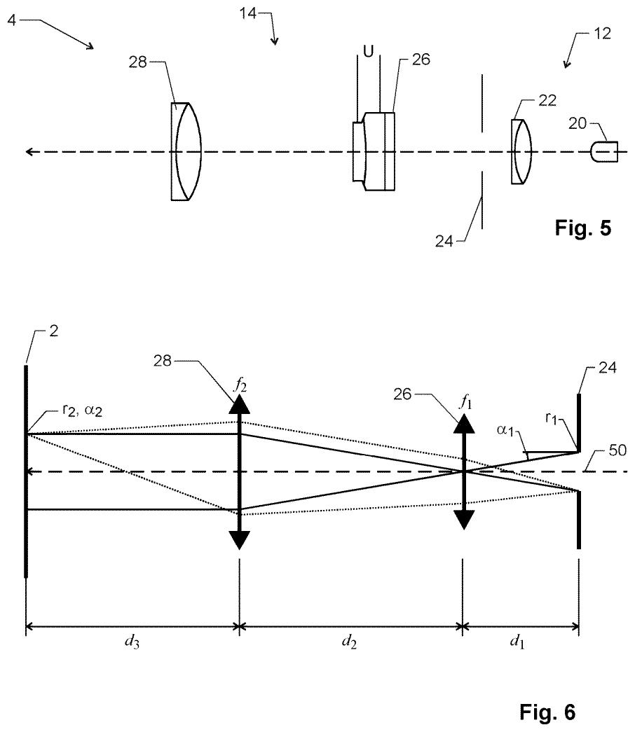

Projection system 4 is shown in more detail in FIGS. 3-5.

As mentioned, it comprises a light source assembly 12 for generating a defined light spot. In the present embodiment, light source assembly 12 comprises a light source 20 generating white light. Light source 20 may be an white-light LED. The light from light source 20 is cast, by means of a lens element 22, onto a field stop 24. These components advantageously form a Kohler-type illumination as it is known to the skilled person.

The light intensity at field stop 24 is advantageously homogeneous in the sense that it varies, over the transparent region of field stop 24, by less than 20%.

Field stop 24 is advantageously an adjustable diaphragm or a revolver-type mechanism with several diaphragms of different diameters on a revolving carrier, with control unit 18 being adapted to vary its diameter as required by the current examination. This allows presenting stimulus light spots of various size to the patient.

Projection optics 14 is adapted to project field stop 24 onto screen 2, i.e. field stop 24 and the stimulus light spot on screen 2 are, in respect to projection optics 14, at conjugate planes, such that the stimulus light spot on screen 2 is an image of field spot 24 and therefore has a well-defined diameter.

In the present embodiment, projection optics 14 comprises an adjustable lens 26 and a projection lens element 28, with the adjustable lens 26 being arranged between light source assembly 12 and projection lens element 28.

Adjustable lens 26 has an adjustable focal length, which can e.g. be controlled by means of a control voltage U or control current I generated by control unit 18.

Adjustable lens 26 may be a controllable liquid lens, in which the curvature of an interface of a liquid can be varied by means of an applied electric field or by mechanical means. An example of a field-controllable liquid lens is described in EP167892A1, where the curvature of the interface between two non-mixing liquids can be varied by means of the electric field.

In another embodiment, a controllable liquid lens such as described in EP2860555A1 may be used.

In yet another embodiment, a lens using electroactive polymers may be used, such as e.g. described in U.S. Pat. No. 10,007,034.

For example, adjustable lens 26 may be a variable focus liquid lens of Corning's A-series.

Advantageously, adjustable lens 26 contains no mechanical motors for silent operation.

Projection lens element 28 is a positive lens element, i.e. it is a converging lens element.

Projection system 4 may further comprise an auxiliary mirror 30 casting the light from projection optics onto adjustable deflector 16 if adjustable deflector 16 is not arranged coaxially to adjustable lens 26 and projection lens element 28.

### Adjustable Deflector

Adjustable deflector 16 is best shown in FIGS. 3 and 4.

In the present embodiment, it comprises a tiltable mirror 36, which can be tilted about a first and a second axis A1, A2. As mentioned above, axes A1 and A2 are, transversal, and they intersect. Advantageously, they perpendicular to each other.

A first and a second rotatory actuator 38-1, 38-2 are provided for tilting mirror 36. They may e.g. be stepper motors, and their rotational position can be controlled by control unit 18.

First rotatory actuator 38-1 has a first shaft 40-1 that can be rotated about first axis A1. First shaft 40-1 is connected, via a first coupler 42-1, to mirror 36. First coupler 42-1 rotationally locks the mirror to first shaft 40-1 for rotations about first axis A1, but it allows mirror 36 to tilt about a third axis A3 transversal to first axis A1. Third axis A3 can advantageously be pivoted, by rotation about first axis A1, to be parallel to second axis A2.

In the present embodiment, first coupler 42-1 comprises a first arm 44-1a rigidly connected to first shaft 40-1 and a first arcuate section 44-1b rigidly connected to first arm 44-1a. First arcuate section 44-1b is connected, by one or two rotational bearings 46-1, to mirror 36.

Second rotatory actuator 38-2 has a second shaft 40-2 that can be rotated about second axis A2. Second shaft 40-2 is connected, via a second coupler 42-2, to mirror 36. Second coupler 42-2 rotationally locks the mirror to second shaft 40-2 for rotations about second axis A2, but it allows mirror 36 to tilt about a fourth axis A4 transversal to second axis A2. Fourth axis A4 can advantageously be pivoted, by rotation about second axis A2, to be parallel to first axis A1.

In the present embodiment, second coupler 42-2 comprises a second arcuate section 44-2 rigidly connected to second shaft 40-2. Second arcuate section 44-2 is connected, by one or two rotational bearings 46-2, to mirror 36.

The first and second axes A1, A2 are advantageously perpendicular to each other in order to obtain the best angular resolution of mirror 36.

As shown in FIG. 4, they are advantageously extending symmetrically to a plane 48 of screen axis 10, i.e. to a plane in which screen axis 10 lies.

As shown in FIG. 2, mirror 36 can be tilted, by means of the rotatory actuators 38-1, 38-2, to guide the light from projection optics 14 to various locations L, L′ on screen 2.

Mirror 36 is radially offset from center axis 10 of screen 2. Since it is therefore not at the center of the spherical screen surface, the path length of the light from projection optics 14 to screen 2 is non-constant and a function the location L, L′. For this reason, the focal length of adjustable lens 26 is adapted as described in more detail in the next section.

As can also be seen in FIG. 2, first and second actuator 38-1, 38-2 are arranged, in respect to center axis 10 of screen 2, radially outward from mirror 36 (i.e. they are further away from center axis 10 than mirror 36) in order to avoid interfering with the patient's field of view.

### Adjusting for Path Length

As mentioned in the previous section, the path length of the light to screen 2 is non-constant and a function the location L, L′.

For this reason, control unit 18 controls the focal length of adjustable lens 26 as a function of the location L, L′ in order to generate a well-focused stimulus light spot on screen 2 for all locations L, L′. In addition, projection optics 14 is adapted to have a magnification that is independent of the location L, L′ and the setting of the focal length of adjustable lens 26. This is explained in more detail in the following.

The present section refers to FIG. 6, where the projection of field stop 24 onto screen 2 by means of adjustable lens 26 and projection lens component 28 is shown schematically, with the optical axis 50 of the system shown as a straight line (in reality, optical axis 50 is bent at the two mirrors 30, 36). The following parameters are used to describe the system:
f₁: The focal length of adjustable lens 26. This length is varied by control unit 18 as a function of the location L of the stimulus light spot on screen 2.
f₂: The focal length of projection lens element 28. This length is advantageously fixed.
d₁: The axial distance between field stop 24 and adjustable lens 26. This distance is advantageously fixed.
d₂: The axial distance between adjustable lens 26 and projection lens element 28. This distance is advantageously fixed.
d₃: The axial distance between projection lens element 28 and screen 2. This distance is a function of the location L.

In the following, the projection system is approximated by means of ray transfer matrix analysis, where the individual elements are described by their ray transfer matrix.

As known to the skilled person, a ray transfer matrix (or ABCD-matrix) describes how an element maps radial locations and beam angles between its input side and its output side, see e.g. https://en.wikipedia.org/wiki/Ray_transfer_matrix_analysis.

The projection system of FIG. 6 maps a beam angle α₁ and a radial location r₁ at its input (in the plane of field stop 24) to a beam angle α₂ and a radial location r₂ at its output (on screen 2) as follows:

$$[ \begin{matrix}{r}_{2} \\ {α}_{2}\end{matrix} ] = [ \begin{matrix}1 & {d}_{3} \\ 0 & 1\end{matrix} ] \cdot [ \begin{matrix}1 & 0 \\ - 1 / {f}_{2} & 1\end{matrix} ] \cdot [ \begin{matrix}1 & {d}_{2} \\ 0 & 1\end{matrix} ] \cdot [ \begin{matrix}1 & 0 \\ - 1 / {f}_{1} & 1\end{matrix} ] \cdot [ \begin{matrix}1 & {d}_{1} \\ 0 & 1\end{matrix} ] \cdot [ \begin{matrix}{r}_{1} \\ {α}_{1}\end{matrix} ] \qquad ( 1 )$$

The matrices on the right-hand side yield, when multiplied, yield

$$[ \begin{matrix}A & B \\ C & D\end{matrix} ] = [ \begin{matrix}\frac{\begin{matrix}{d}_{2} ( {d}_{3} - {f}_{2} ) - \\ {d}_{3} ( {f}_{1} + {f}_{2} ) + {f}_{1} \cdot {f}_{2}\end{matrix}}{{f}_{1} \cdot {f}_{2}} & \begin{matrix}\frac{\begin{matrix}{d}_{1} ( {d}_{2} ( {d}_{3} - {f}_{2} ) - \\ {d}_{3} ( {f}_{1} + {f}_{2} ) + {f}_{1} \cdot {f}_{2} )\end{matrix}}{{f}_{1} \cdot {f}_{2}} + \\ {d}_{2} ( 1 - \frac{{d}_{3}}{{f}_{2}} ) + {d}_{3}\end{matrix} \\ \frac{{d}_{2} - {f}_{1} - {f}_{2}}{{f}_{1} \cdot {f}_{2}} & \frac{{d}_{1} ( {d}_{2} - {f}_{1} - {f}_{2} )}{{f}_{1} \cdot {f}_{2}} - \frac{{d}_{2}}{{f}_{2}} + 1\end{matrix} ] \qquad ( 2 )$$

B in Eq. (2) must be zero if the output plane is to be the conjugate of the input plane as desired, i.e.

$$\frac{{d}_{1} ( {d}_{2} ( {d}_{3} - {f}_{2} ) - {d}_{3} ( {f}_{1} + {f}_{2} ) + {f}_{1} \cdot {f}_{2} )}{{f}_{1} \cdot {f}_{2}} + {d}_{2} ( 1 - \frac{{d}_{3}}{{f}_{2}} ) + {d}_{3} = 0 , \qquad ( 3 )$$

This yields, for the focal length f₁ of adjustable lens 26:

$${f}_{1} = \frac{{d}_{1} ( {d}_{2} ( {d}_{3} - {f}_{2} ) - {d}_{3} \cdot {f}_{2} )}{{d}_{1} ( {d}_{2} - {f}_{2} ) + {d}_{2} ( {d}_{3} - {f}_{2} ) - {d}_{3} \cdot {f}_{2}} \qquad ( 4 )$$

The values of f₂ as well as d₁ and d₂ are constant and known. The distance d₃ between projection lens element 28 and screen 2 along the optical axis can be calculated from the positions of lens element 28, the mirrors 30, 36, screen 2, and the current spot location L, L′. Hence, control unit 18 can calculate the focal length f₁ of adjustable lens 26 from Eq. (4) or from a suitable look-up-table, and it can set it accordingly, thereby ensuring that field stop 24 is projected, in focus, onto screen 2.

A in Eq. (2) is the magnification factor between the input plane and the output plane, i.e. between the field stop and the screen:

$$A = \frac{{d}_{2} ( {d}_{3} - {f}_{2} ) - {d}_{3} \cdot {f}_{2}}{{d}_{1} \cdot {f}_{2}} \qquad ( 5 )$$

As can be seen, magnification factor A generally depends on the distance d₃. Hence, for most of the values of f₂, d₁, and d₂, control unit 18 would need to adapt the diameter of field stop 24 as a function of the current spot location L, L′ to keep the spot size on screen 2 constant, and it would also have to adapt the brightness of light source 20 in order to keep the spot brightness constant. Both these measures may be used, but they are advantageously avoided.

Hence, in an advantageous embodiment, the magnification A is chosen such that it is independent on the distance d₃, i.e. the derivative of A in respect to

$$\frac{\partial A}{\partial {d}_{3}} = \frac{\partial }{\partial {d}_{3}} \frac{{d}_{2} ( {d}_{3} - {f}_{2} ) - {d}_{3} \cdot {f}_{2}}{{d}_{1} \cdot {f}_{2}} = 0 , \qquad ( 6 )$$

$$i . e .$$

$$\frac{{d}_{2} - {f}_{2}}{{d}_{1} \cdot {f}_{2}} = 0. \qquad ( 7 )$$

Hence, if the focal length f₂ of projection lens element 28 is chosen to be equal to the distance d₂ between projection lens element 28 and adjustable lens 26, the magnification A of the projection optics becomes independent of the distance d₃.

Therefore, advantageously, f₂ is chosen to be equal to d₂. In this case, control unit 18 merely needs to maintain the focal length f₁ as given by Eq. (4) in order to generate a well-focused stimulus light spot of constant size and brightness at all locations L, L′.

There is no need for the condition f₂=d₂ to be fulfilled exactly. Small deviations will lead to variations in brightness and stimulus spot size. According to perimetry ISO norm EN ISO 12866:1999 (Ophthalmic instruments Perimeters), brightness is allowed to vary within a range of −20% to +25%, and spot size within a range of −15% to +20%. Hence, advantageously, the focal length f₂ of the projection lens element is equal, within ±20% of f₂, to the axial distance d₂.

In operation, over the used locations L, L′, the distance d₃ will vary between a minimum value d_{3,min} and a maximum value d_{3,max}.

The values of d_{3,min}, d_{3,max} depend on device geometry. Perimeters often use a Goldmann-type screen with a radius of R=300 mm (see FIG. 2). As can be calculated for a geometry of the type of FIG. 2, where the location of mirror 36 is offset by roughly 100 mm from center axis 10 and where the incoming light has angle α of approximately 600 in respect to center axis 10, distance d₃ typically varies between 550 and 750 mm.

Adjustable lenses, e.g. of the type mentioned above, are usually best operated at low curvature, i.e. at large or infinite focal length, i.e. f₁=∞.

In other words, the present method advantageously includes the step of setting the focal length of adjustable lens 26 to infinity for at least some of the locations L, L′.

f₁=∞ is the case when the denominator of Eq. (4) is zero. The corresponding value for the distance d₃ is

$${d}_{3 , inf} = \frac{{f}_{2} ( {d}_{1} + {d}_{2} )}{{d}_{1} + {d}_{2} - {f}_{2}} \qquad ( 8 )$$

Hence, advantageously, the components of the perimeter are placed such that d_{3,inf} is between d_{3,min} and d_{3,max}, i.e.

$${d}_{3 , min} < \frac{{f}_{2} ( {d}_{1} + {d}_{2} )}{{d}_{1} + {d}_{2} - {f}_{2}} < {d}_{3 , max} \qquad ( 9 )$$

For the embodiment of Eq. (7), i.e. d₂=f₂ we obtain

$${d}_{3 , inf} = \frac{{d}_{2} ( {d}_{1} + {d}_{2} )}{{d}_{1}} \qquad ( 10 )$$

In other words, the components are advantageously placed such that, d₁, d₂, d_{3,min}, d_{3,max} fulfill

$${d}_{3 , min} < \frac{{d}_{2} ( {d}_{1} + {d}_{2} )}{{d}_{1}} < {d}_{3 , max} \qquad ( 11 )$$

The corresponding range of focal lengths f₁, in order to accommodate for d₃=d_{3,min}=550 mm and d₃=d_{3,max}=750 mm, for values of d₁=50 mm and d₂=160 mm and assuming that d₂=f₂, are f₁=200 mm (+5 diopters) and f₁=−328 mm (−3 diopters) respectively, i.e. a tuning range of roughly 8 diopters is required in this case for adjustable lens 26.

### Notes

In the embodiment above, the deflector only has a single mirror tiltable around two axes. Alternatively, two separately tiltable mirrors may be used.

In yet another embodiment, there may be no tiltable mirror, or the mirror may be tiltable only about a single axis. In this case, the beam deflector can be implemented, along at least one or both directions, by means of a pivot mechanism tilting the light source assembly and/or the projection optics.

The at least one mirror of the adjustable deflector is advantageously a flat mirror. However, it may also be a curved mirror, or mirror 30 may be curved, in which case the mirror is considered to form part of the projection optics, in particular of the projection lens element.

In the example above, the projection lens element 28 is a lens. Alternatively, it may also be formed by a curved mirror alone, in particular the curved mirror of the adjustable deflector.

While there are shown and described presently preferred embodiments of the invention, it is to be distinctly understood that the invention is not limited thereto but may be otherwise variously embodied and practiced within the scope of the following claims.

1. A perimeter comprising:
a screen,
a light source assembly,
projection optics adapted to process the light from the light source and to project a stimulus light spot onto the screen, and
an adjustable deflector,
wherein the projection optics comprises an adjustable lens with adjustable focal length.

2. The perimeter of claim 1, wherein the projection optics comprises a projection lens element arranged coaxially with and at a distance from the adjustable lens.

3. The perimeter of claim 2, wherein the projection lens element is a positive lens element.

4. The perimeter of claim 3, wherein a focal length of the projection lens element is equal, within ±20%, to an axial distance between the adjustable lens and the projection lens element.

5. The perimeter of claim 2, wherein the adjustable lens is arranged between the light source assembly and the projection lens element.

6. The perimeter of claim 1, wherein
$${d}_{3 , min} < \frac{{f}_{2} ( {d}_{1} + {d}_{2} )}{{d}_{1} + {d}_{2} - {f}_{2}} < {d}_{3 , max} ,$$
with
d₁ being an axial distance between a field stop of the light source assembly and the adjustable lens,
d₂ being an axial distance between the adjustable lens and the projection lens element,
f₂ being the focal length of the projection lens element, and
d_{3,min}, and d_{3,max} being the smallest and largest possible axial distances between the projection lens element and the screen.

7. The perimeter of claim 6,
wherein the projection lens element is a positive lens element, wherein a focal length of the projection lens element is equal, within ±20%, to an axial distance between the adjustable lens and the projection lens element, and
wherein
$${d}_{3 , min} < \frac{{d}_{2} ( {d}_{1} + {d}_{2} )}{{d}_{1}} < {d}_{3 , max} .$$

8. The perimeter of claim 1, wherein an aperture of the projection optics and the deflector is selected to be sufficiently large for any light beam from the light source assembly traversing the adjustable lens to arrive at the screen.

9. The perimeter of claim 1, wherein the light source assembly comprises a field stop, wherein the projection optics projects the field stop onto the screen.

10. The perimeter of claim 1 comprising a control unit connected to the adjustable lens and the adjustable deflector and being adapted to
positioning a stimulus light spot on the screen at a plurality of locations by adjusting the adjustable deflector, wherein an optical path length from the light source assembly to the location varies between at least some of said locations, and
adjusting the focal length of the adjustable lens as a function of the path length.

11. The perimeter of the claim 10,
wherein the light source assembly corn rises a field stop,
wherein the projection optics projects the field stop onto the screen, and
wherein the control unit is adapted to adjusting the focal length of the adjustable lens such that, for all said locations, the projection optics projects the field stop onto the screen.

12. The perimeter of claim 11, wherein the projection optics has the same magnification for all said locations.

13. The perimeter of claim 1, wherein the adjustable deflector comprises a mirror radially offset from a center axis of the screen.

14. The perimeter of claim 1, wherein said adjustable deflector comprises a mirror tiltable around first and second axes, with the first and second axes being transversal to each other.

15. The perimeter of claim 14, wherein the adjustable deflector further comprises
a first rotatory actuator with a first shaft rotatable around first axis,
a second rotatory actuator with a second shaft rotatable around the second axis,
a first coupler coupling the mirror and the first shaft, wherein the first coupler rotationally locks the mirror to the first shalt for rotations about the first axis but allows the mirror to tilt about a third axis transversal to the first axis,
a second coupler coupling the mirror to the second shaft, wherein the second coupler rotationally locks the mirror to the second shaft for rotations about the second axis but allows the mirror to tilt about a fourth axis transversal to the second axis.

16. The perimeter of claim 15, wherein the first and second actuator are arranged, in respect to the center axis of the screen radially outward from the mirror.

17. The perimeter of claim 14, wherein the first and second axes are symmetric to a plane of the screen axis.

18. The perimeter claim 1, wherein the adjustable lens is a controllable liquid lens.

19. A method for operating a perimeter that comprises
a screen,
a light source assembly,
projection optics, and
an adjustable deflector,
wherein the projection optics comprises an adjustable lens with adjustable focal length,
the method comprising:
by the projection optics, processing the light from the light source project a stimulus light spot onto the screen, and
positioning a stimulus light spot on the screen at a plurality of locations by adjusting the adjustable deflector.

20. The method of claim 19, wherein an optical path length from the light source assembly to the location varies between at least some of said locations and wherein the method comprises
adjusting the focal length of the adjustable lens has a function of the path length.

21. The method of claim 20, further comprising adjusting the focal length of the adjustable lens such that, for all said locations the projection optics projects a field stop of the light source assembly onto the screen.

22. The method of claim 19, comprising setting the focal length of the adjustable lens to infinity for at least some of the locations.
